(19) United States
(12) Patent Application Publication
(10) Pub. No.: US 2025/0009307 A1
(43) Pub. Date: Jan. 9, 2025

(54) DENOISING ENCODER-DECODER NEURAL NETWORK FOR PAIN RECOGNITION AND OTHER DIAGNOSTIC APPLICATIONS

(71) Applicant: Cornell University, Ithaca, NY (US)

(72) Inventors: Hayford Agbanu Perry Fordson, Ithaca, NY (US); Adam K. Anderson, Ithaca, NY (US); Eve DeRosa, Ithaca, NY (US)

(21) Appl. No.: 18/765,920

(22) Filed: Jul. 8, 2024

Related U.S. Application Data
(60) Provisional application No. 63/525,520, filed on Jul. 7, 2023.

(51) Int. Cl. A61B 5/00; A61B 5/024; A61B 5/0245; A61B 5/308; A61B 5/318; G16H 40/67

(52) U.S. Cl. CPC A61B 5/7264; A61B 5/0006; A61B 5/02416; A61B 5/0245; A61B 5/308; A61B 5/318; G16H 40/67

(57) ABSTRACT

A method in an illustrative embodiment comprises obtaining an input data signal for a given individual, generating a noisy version of the input data signal, processing the noisy version of the input data signal in a denoising encoder-decoder neural network to generate a classification for the input data signal, and executing at least one automated action based at least in part on the generated classification. The automated action may comprise, for example, a remedial action, or another type of action. In some embodiments, the input data signal comprises at least one ECG data signal from at least one ECG sensor and/or at least one PPG data signal from at least one PPG sensor, although additional or alternative data signals can be used. The classification for the input data signal in some embodiments comprises, for example, a pain recognition classification providing a pain biomarker for the input data signal.

### Related Application

The present application claims priority to U.S. Provisional Patent Application Ser. No. 63/525,520, filed Jul. 7, 2023 and entitled “Denoising Encoder-Decoder Neural Network for Pain Recognition and Other Diagnostic Applications,” which is incorporated by reference herein in its entirety.

### FIELD

The field relates generally to information processing systems, and more particularly to machine learning and other types of artificial intelligence implemented in such systems.

### BACKGROUND

Pain is a subjective state created by the brain. About 1.5 billion people world-wide experience acute or chronic pain each day. Physicians often rely on patients' subjective self-reports on pain sensations, as reliable objective measurements are typically unavailable. However, there are many individuals who cannot verbally or accurately report their pain due to anesthesia, post-operative conditions, coma, cognitive impairment, interoceptive impairment, dementia and other conditions. Additionally, some individuals may over-report their pain levels in order to obtain access to pharmaceuticals. A need therefore exists for improved techniques for accurate and efficient diagnosis in pain recognition and other diagnostic applications.

### SUMMARY

Illustrative embodiments disclosed herein implement neural networks for pain recognition and other diagnostic applications. For example, some embodiments provide a deep artificial denoising auto-encoder-decoder (DADAED) neural network or other type of denoising encoder-decoder neural network that advantageously provides superior performance relative to conventional approaches to pain recognition and other patient diagnostics.

These and other embodiments can be implemented as low-cost, accurate and automatic diagnostic systems that utilize, for example, low resolution electrocardiogram (ECG) data and/or photoplethysmography (PPG) data to improve pain recognition or other patient diagnostics, thereby providing a vital resource for healthcare and medical research.

Some embodiments provide objective pain biomarkers utilizing data signals associated with the heart-brain axis. The brain regulates the heart via the autonomous nervous system (ANS), reflecting complex mental states like emotions and stress, but also specifically injury to the body. Illustrative embodiments disclosed herein, by performing pain recognition from data signals associated with the heart-brain axis, provide automated, objective assessment of pain levels experienced by patients who might not otherwise be capable of accurately self-reporting their pain levels.

The disclosed techniques are highly accurate and efficient, and provide substantial improvements relative to conventional approaches, not only for pain recognition but in a wide variety of different medical contexts, such as classification of emotional distress, as well as numerous other processing contexts.

One or more such embodiments illustratively further provide various types of automated remediation responsive to pain recognition outputs or other types of outputs of denoising encoder-decoder neural network. For example, some embodiments implement classification and remediation algorithms to at least partially automate various aspects of patient care in healthcare applications such as telemedicine. Such applications can involve a wide variety of different types of remote medical monitoring and intervention.

In an illustrative embodiment, a method comprises obtaining an input data signal for a given individual, generating a noisy version of the input data signal, processing the noisy version of the input data signal in a denoising encoder-decoder neural network to generate a classification for the input data signal, and executing at least one automated action based at least in part on the generated classification. The automated action may comprise, for example, a remedial action, or another type of action.

In some embodiments, the input data signal comprises at least one ECG data signal from at least one ECG sensor and/or at least one PPG data signal from at least one PPG sensor, although additional or alternative data signals can be used.

The classification for the input data signal in some embodiments comprises, for example, a pain recognition classification providing a pain biomarker for the input data signal. Additional or alternative types of diagnostic classifications can be similarly generated using the disclosed techniques, to provide other types of biomarkers.

In some embodiments, executing at least one automated action based at least in part on the generated classification illustratively comprises generating at least one output signal in a telemedicine application. For example, such output signals in a telemedicine application can comprise classification information for presentation on a user terminal or other display device, classification information transmitted over a network to a medical professional, and/or classification information transmitted over a network to a prescription-filling entity. A wide variety of other signals can be generated in conjunction with execution of one or more automated actions in illustrative embodiments.

It is to be appreciated that the foregoing arrangements are only examples, and numerous alternative arrangements are possible.

These and other illustrative embodiments include but are not limited to systems, methods, apparatus, processing devices, integrated circuits, and computer program products comprising processor-readable storage media having software program code embodied therein.

### BRIEF DESCRIPTION OF THE FIGURES

FIG. 1 shows an information processing system comprising a processing platform implementing a denoising encoder-decoder neural network in an illustrative embodiment.

FIGS. 2 and 3 show example implementations of denoising encoder-decoder neural networks in illustrative embodiments.

FIG. 4 shows a plot of an example signal segmentation and pain quantification configuration in an illustrative embodiment.

FIGS. 5 and 6 show performance data generated for experiments performed on illustrative embodiments.

FIG. 7 shows another example implementation of a machine learning system in an illustrative embodiment.

### DETAILED DESCRIPTION

Illustrative embodiments can be implemented, for example, in the form of information processing systems comprising one or more processing platforms each having at least one computer, server or other processing device. A number of examples of such systems will be described in detail herein. It should be understood, however, that embodiments of the invention are more generally applicable to a wide variety of other types of information processing systems and associated computers, servers or other processing devices or other components. Accordingly, the term “information processing system” as used herein is intended to be broadly construed so as to encompass these and other arrangements. Moreover, the particular embodiments described herein are presented by way of illustrative example only, and should not be construed as limiting in any way.

Some embodiments disclosed herein advantageously provide low-cost, accurate and automatic pain recognition systems that utilize low resolution ECG data and/or PPG data to improve pain recognition in healthcare, medical research and other applications. Such arrangements provide significant improvements over conventional approaches that typically require high-resolution, invasive and expensive equipment to collect data, and generally rely on patients' subjective assessment of pain sensation.

For example, some embodiments provide deep artificial denoising auto-encoder-decoder (DADAED) neural networks that process low resolution ECG data and/or PPG data to assess pain threshold and pain tolerance. Experiments performed on illustrative embodiments utilizing the BioVid Heat Pain Database are described herein and demonstrate significant improvements relative to conventional approaches. Other types of neural networks or more generally machine learning systems can be implemented in other embodiments.

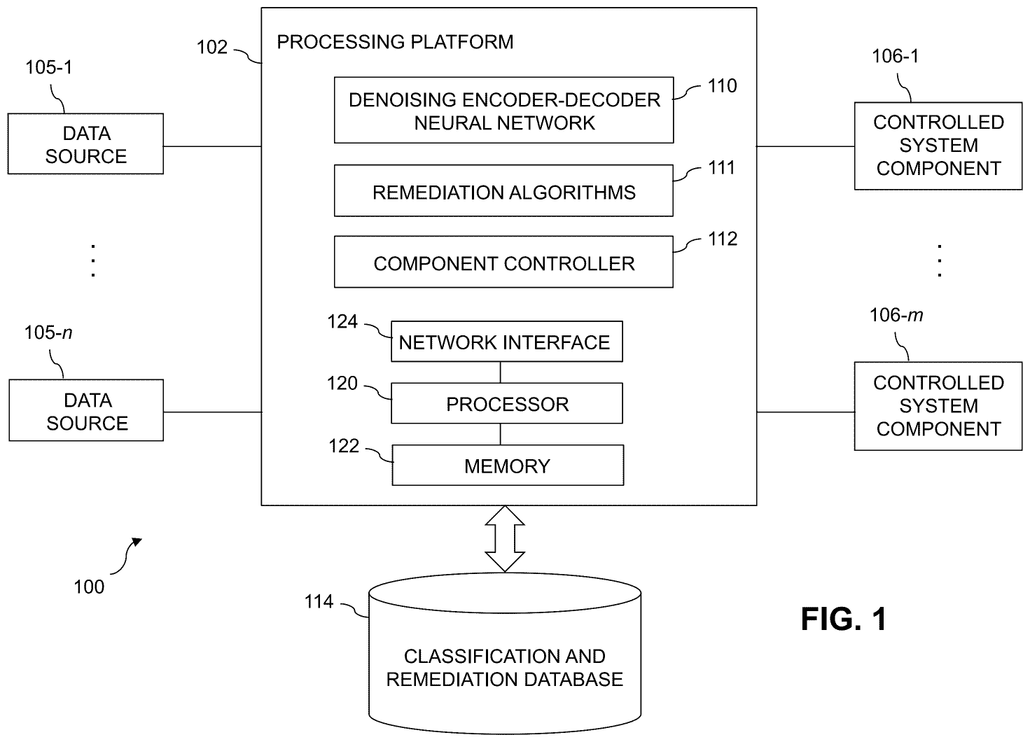

FIG. 1 shows an information processing system 100 implementing a machine learning and/or artificial intelligence (AI) system comprising at least one neural network adapted to classify input data in one or more designated processing contexts, such as pain recognition. The system 100 comprises a processing platform 102. Coupled to the processing platform 102 are data sources 105-1, . . . 105-n and controlled system components 106-1, . . . 106-m, where n and m are arbitrary integers greater than or equal to one and may but need not be equal. Accordingly, some embodiments can include only a single data source and/or only a single controlled system component. For example, a data source can comprise a single-lead sensor or a multi-dimensional sensor array, and the controlled system component can comprise an alert or notification generator or other information display for presenting information relating to a classification output. Numerous alternative arrangements are possible.

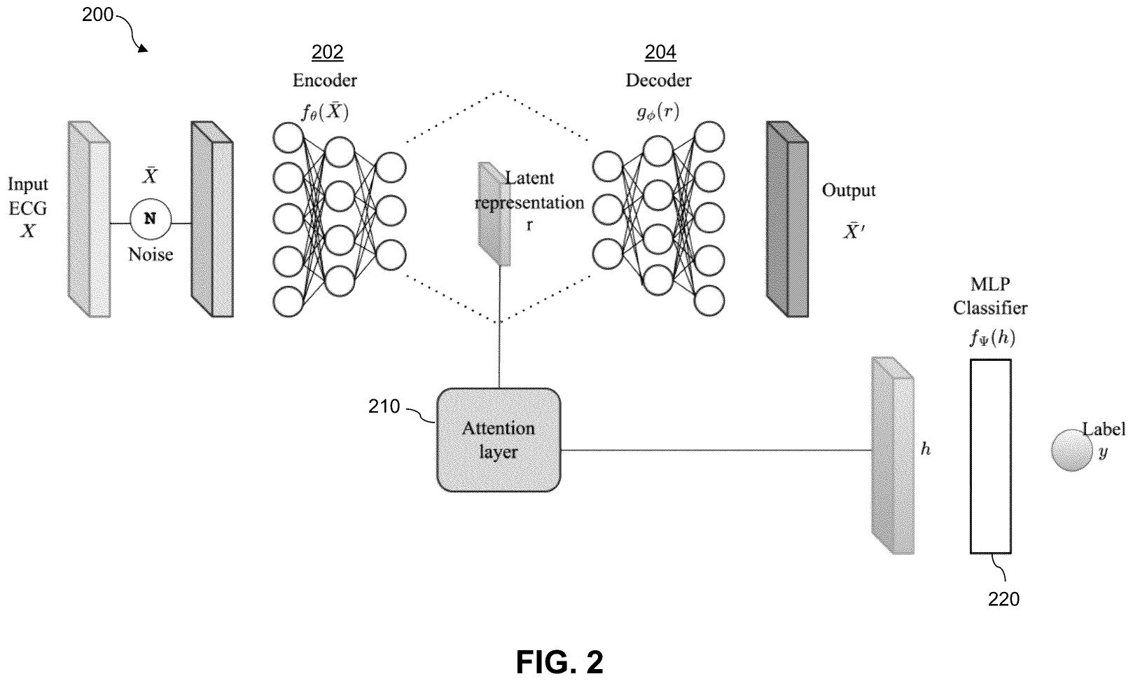

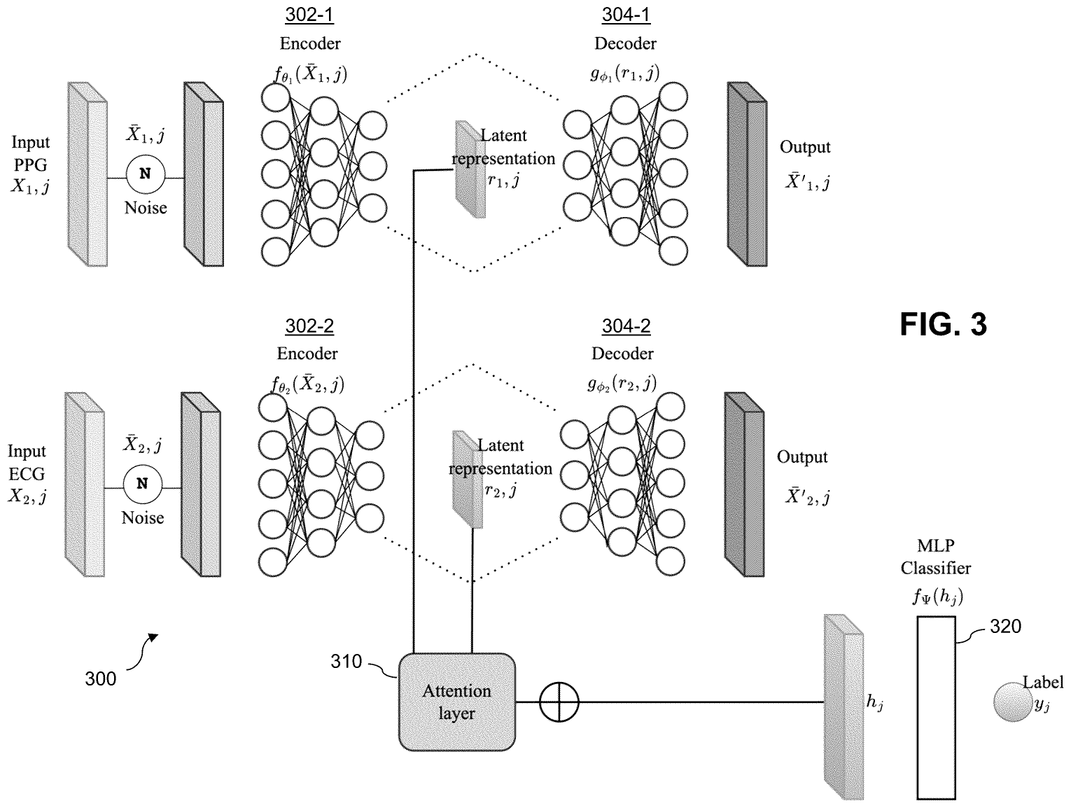

The processing platform 102 implements at least one neural network 110, illustratively a denoising encoder-decoder neural network (e.g., a DADAED neural network) as disclosed herein, as well as one or more remediation algorithms 111, and at least one component controller 112. The neural network 110 in the present embodiment illustratively implements an AI-based classification algorithm to classify input data signals such as ECG signals and/or PPG signals in applications such as pain recognition, although numerous other arrangements are possible. The DADAED neural network, illustrative implementations of which are shown in FIGS. 2 and 3, is an example of what is more generally referred to herein as a “denoising encoder-decoder neural network.” A wide variety of other types and arrangements of neural networks can be used in illustrative embodiments, and the term “neural network” as used herein is therefore intended to be broadly construed.

In operation, the processing platform 102 is illustratively configured to obtain an input data signal for a given individual, such as a patient undergoing diagnosis or treatment, to generate a noisy version of the input data signal, to process the noisy version of the input data signal in the neural network 110, which is illustratively a denoising encoder-decoder neural network, such as a DADAED neural network of the type shown in FIG. 2 or FIG. 3, to generate a classification for the input data signal, and to execute under the control of at least one of the remediation algorithms 111 at least one automated action based at least in part on the generated classification, illustratively via the component controller 112.

Different ones of the remediation algorithms 111 are illustratively configured to provide different automated remedial actions for different classification outcomes. For example, some embodiments activate different ones of the controlled system components in different ways via the component controller 112 based on different classification outcomes generated by the neural network 110.

The term “remedial action” as used herein is intended to be broadly construed, so as to encompass any type of action that attempts to address, correct or otherwise respond to a particular classification outcome. For example, a remedial action may involve presenting information associated with the classification outcome to a medical professional for use in diagnosing a patient.

As another example, a remedial action may comprise generating an alert and sending such an alert over a network. A wide variety of other types of remedial actions can be performed. Also, other types of automated actions not necessarily involving remediation can be performed responsive to particular classification outcome.

In some embodiments, the data sources 105 can comprise, for example, one or more internal devices of the given individual, one or more wearable devices of the given individual, a smartphone of the given individual, and/or one or more other types of sensors associated with the given individual, in any combination.

The generated classification can comprise, for example, an indicator of a particular detected physiological condition of the given individual, such as a particular level of pain currently being experienced, although a wide variety of other types of classifications can be generating using the neural network 110 in other embodiments.

An input data signal applied to the processing platform 102 illustratively comprises, for example, at least one electrocardiogram (ECG) data signal obtained from one or more ECG sensors and/or at least one photoplethysmography (PPG) data signal obtained from one or more PPG sensors. Illustrative embodiments of this type will be described in more detail below with reference to FIGS. 2 and 3. More particularly, the illustrative embodiment of FIG. 2 processes an input ECG data signal, and the illustrative embodiment of FIG. 3 processes both an input ECG data signal and an input PPG data signal.

Other types of data signals characterizing one or more physiological conditions or other biomedical conditions of the given individual may be used.

Accordingly, in some embodiments, the neural network 110 is configured to process multiple data signals of different types.

For example, the input data signal may comprise at least a first data signal of a first type and a second data signal of a second type different than the first type, with noisy versions of the respective first and second data signals being generated and applied to respective first and second encoder-decoder pairs of the neural network 110 for generation of respective first and second latent representations therefrom, with the first and second latent representations being processed to generate the classification for the input data signal. Illustrative embodiments of this type will be described in more detail below with reference to FIG. 3.

In a more particular example of such an embodiment, the first data signal of the first type illustratively comprises at least one ECG data signal and the second data signal of the second type illustratively comprises at least one PPG data signal, as shown in the FIG. 3 embodiment, although a wide variety of different combinations of multiple distinct signal types may be used in other embodiments.

In some embodiments, the classification for the input data signal comprises a pain recognition classification providing a pain biomarker for the input data signal. Such pain biomarkers are also referred to herein as AI-based pain biomarkers.

Numerous other arrangements of system components and associated generated classifications are possible.

It is to be appreciated that the term “neural network” as used herein is intended to be broadly construed to encompass a wide variety of different types of processor-based machine learning and/or artificial intelligence arrangements. Such a neural network is implemented by at least one processing device comprising a processor coupled to a memory.

The component controller 112 generates one or more control signals for adjusting, triggering or otherwise controlling various operating parameters associated with the controlled system components 106 based at least in part on classifications generated by the neural network 110 and processed by one or more of the remediation algorithms 111. A wide variety of different type of devices or other components can be controlled by component controller 112, possibly by applying control signals or other signals or information thereto, including additional or alternative components that are part of the same processing device or set of processing devices that implement the processing platform 102. Such control signals, and additionally or alternatively other types of signals and/or information, can be communicated over one or more networks to other processing devices, such as user terminals associated with respective system users.

The processing platform 102 is configured to utilize a classification and remediation database 114. Such a database illustratively stores user data, user profiles and a wide variety of other types of information, including data from one or more of the data sources 105, that may be utilized by the neural network 110 in performing classification and remediation operations. The classification and remediation database 114 is also configured to store related information, including various processing results, such as classifications or other outputs generated by the neural network 110.

The component controller 112 utilizes outputs generated by the neural network 110 and/or one or more of the remediation algorithms 111 to control one or more of the controlled system components 106. The controlled system components 106 in some embodiments therefore comprise system components that are driven at least in part by outputs generated by the neural network 110. For example, a controlled component can comprise a processing device such as a computer, a smartphone, a wearable device, an internal device, an intelligent medical monitoring device, a handheld sensor device or other type of processing device that presents a display to a user and/or directs a user to respond in a particular manner responsive to an output of a classification algorithm. These and numerous other different types of controlled system components 106 can make use of outputs generated by the neural network 110, including various types of equipment and other systems associated with one or more of the example use cases described elsewhere herein.

Although the neural network 110, remediation algorithms 111 and the component controller 112 are all shown as being implemented on processing platform 102 in the present embodiment, this is by way of illustrative example only. In other embodiments, the neural network 110, remediation algorithms 111 and the component controller 112 can each be implemented on a separate processing platform, or using other arrangements. A given such processing platform is assumed to include at least one processing device comprising a processor coupled to a memory.

Examples of such processing devices include computers, servers or other processing devices arranged to communicate over a network. Storage devices such as storage arrays or cloud-based storage systems used for implementation of classification and remediation database 114 are also considered “processing devices” as that term is broadly used herein.

The network can comprise, for example, a global computer network such as the Internet, a wide area network (WAN), a local area network (LAN), a satellite network, a telephone or cable network, a cellular network such as a 4G or 5G network, a wireless network implemented using a wireless protocol such as Bluetooth, WiFi or WiMAX, or various portions or combinations of these and other types of communication networks.

It is also possible that at least portions of other system elements such as one or more of the data sources 105 and/or the controlled system components 106 can be implemented as part of the processing platform 102, although shown as being separate from the processing platform 102 in the figure.

For example, in some embodiments, the system 100 can comprise a laptop computer, tablet computer or desktop personal computer, a smartphone, a wearable device, an internal device, an intelligent medical monitoring device, a handheld sensor device, or another type of computer or communication device, as well as combinations of multiple such processing devices, configured to incorporate at least one data source and to execute an AI-based classification algorithm for controlling at least one system component.

Examples of automated remedial actions that may be taken in the processing platform 102 responsive to outputs generated by the neural network 110 and/or the remediation algorithms 111 include generating in the component controller 112 at least one control signal for controlling at least one of the controlled system components 106 over a network, generating at least a portion of at least one output display for presentation on at least one user terminal, generating an alert for delivery to at least user terminal over a network, and/or storing the outputs in the classification and remediation database 114.

A wide variety of additional or alternative automated remedial actions may be taken in other embodiments. The particular automated remedial action or actions will tend to vary depending upon the particular use case in which the system 100 is deployed. Other types of automated actions can be performed in other embodiments.

For example, some embodiments implement classification and remediation algorithms to at least partially automate various aspects of patient care in healthcare applications such as telemedicine. Such applications illustratively involve a wide variety of different types of remote medical monitoring and intervention.

An example of an automated remedial action in this particular context includes generating at least one output signal, illustratively comprising at least one of classification information for presentation on a user terminal or other display device, classification information transmitted over a network to a medical professional, and/or classification information transmitted over a network to a pharmacy or other prescription-filling entity. Such classification information can comprise, for example, a classification visualization signal or other type of signal suitable for presentation on a display device.

Additional examples of such use cases are provided elsewhere herein. It is to be appreciated that the term “automated remedial action” as used herein is intended to be broadly construed, so as to encompass the above-described automated remedial actions, as well as numerous other actions that are automatically driven based at least in part on one or more classifications generated using an AI-based classification algorithm as disclosed herein, with such actions being configured to address or otherwise remediate various conditions indicated by the corresponding classifications.

The processing platform 102 in the present embodiment further comprises a processor 120, a memory 122 and a network interface 124. The processor 120 is assumed to be operatively coupled to the memory 122 and to the network interface 124 as illustrated by the interconnections shown in the figure.

The processor 120 may comprise, for example, a microprocessor, an application-specific integrated circuit (ASIC), a field-programmable gate array (FPGA), a central processing unit (CPU), a tensor processing unit (TPU), a graphics processing unit (GPU), an arithmetic logic unit (ALU), a digital signal processor (DSP), or other similar processing device component, as well as other types and arrangements of processing circuitry, in any combination. At least a portion of the functionality of at least one neural network or associated classification and/or remediation algorithm provided by one or more processing devices as disclosed herein can be implemented using such circuitry.

In some embodiments, the processor 120 comprises one or more graphics processor integrated circuits. Such graphics processor integrated circuits are illustratively implemented in the form of one or more GPUs. Accordingly, in some embodiments, system 100 is configured to include a GPU-based processing platform. Such a GPU-based processing platform can be cloud-based configured to implement one or more neural networks for processing data associated with a large number of system users. Other embodiments can be implemented using similar arrangements of one or more TPUs.

Numerous other arrangements are possible. For example, in some embodiments, one or more neural networks and their associated AI-based classification algorithms can be implemented on a single processor-based device, such as a computer, a smartphone, a wearable device, an internal device, an intelligent medical monitoring device, a handheld sensor device or other processing device, utilizing one or more processors of that device. Such embodiments are also referred to herein as “on-device” implementations of AI-based classification algorithms.

The memory 122 stores software program code for execution by the processor 120 in implementing portions of the functionality of the processing platform 102. For example, at least portions of the functionality of neural network 110, remediation algorithms 111 and/or component controller 112 can be implemented using program code stored in memory 122.

A given such memory that stores such program code for execution by a corresponding processor is an example of what is more generally referred to herein as a processor-readable storage medium having program code embodied therein, and may comprise, for example, electronic memory such as SRAM, DRAM or other types of random access memory, flash memory, read-only memory (ROM), magnetic memory, optical memory, or other types of storage devices in any combination.

Articles of manufacture comprising such processor-readable storage media are considered embodiments of the invention. The term “article of manufacture” as used herein should be understood to exclude transitory, propagating signals.

Other types of computer program products comprising processor-readable storage media can be implemented in other embodiments.

In addition, illustrative embodiments may be implemented in the form of integrated circuits comprising processing circuitry configured to implement processing operations associated with one or more of the neural network 110, the remediation algorithms 111 and the component controller 112 as well as other related functionality. For example, at least a portion of the neural network 110 of system 100 is illustratively implemented in at least one neural network integrated circuit of a processing device of the processing platform 102.

The network interface 124 is configured to allow the processing platform 102 to communicate over one or more networks with other system elements, and may comprise one or more conventional transceivers.

It is to be appreciated that the particular arrangement of components and other system elements shown in FIG. 1 is presented by way of illustrative example only, and numerous alternative embodiments are possible. For example, other embodiments of information processing systems can be configured to implement one or more neural networks and associated AI-based classification algorithm and remediation algorithm functionality of the type disclosed herein.

Also, terms such as “data source” and “controlled system component” as used herein are intended to be broadly construed. For example, a given set of data sources in some embodiments can comprise one or more internal devices of an individual, one or more wearable devices of the individual, a smartphone of the individual, and/or one or more other types of sensors associated with the individual.

Additionally or alternatively, data sources can comprise intelligent medical monitoring devices, electrodes, video cameras, sensor arrays or other types of imaging or data capture devices.

Other examples of data sources include various types of databases or other storage systems accessible over a network, where such databases store data signals and other related data. A wide variety of different types of data sources can therefore be used to provide input data to an AI-based classification algorithm in illustrative embodiments. A given controlled component can illustratively comprise a computer, a smartphone, a wearable device, an internal device, an intelligent medical monitoring device, a handheld sensor device or other type of processing device that receives an output from an AI-based classification algorithm and/or an associated remedial algorithm and performs at least one automated remedial action in response thereto.

Example implementations of the neural network 110 will now be described in more detail with reference to FIGS. 2 and 3. In these embodiments, the neural network 110 more particularly comprises a DADAED neural network, although it is to be appreciated that numerous other types and arrangements of neural networks can be used in illustrative embodiments.

FIG. 2 shows one example of a DADAED neural network 200 in an illustrative embodiment. In this embodiment, the DADAED neural network 200 illustratively comprises an encoder-decoder neural network that includes an encoder 202, a decoder 204, an attention layer 210 and a multi-layer perceptron (MLP) classifier 220. In this embodiment, artificial neural networks (ANNs) are utilized to implement the encoder and decoder, although other types and arrangements of neural networks can be used in other embodiments. The DADAED neural network 200 and other neural networks disclosed herein, or portions or combinations thereof, may be viewed as examples of machine learning models, which in some embodiments more particularly comprise deep learning models. Terms such as “neural network,” “machine learning” and “model” as used herein are intended to be broadly construed.

Noise is added to an input ECG data signal and the resulting noisy version of the input ECG data signal is applied to the encoder 202 as shown. The encoder 202 transforms the received signal into a latent representation and then the decoder 204 takes in the latent representation and generates an output. The latent representation generated by the encoder 202 is compressed and lower-dimensional relative to the input ECG data signal. It contains vital features about the input

ECG data signal that are important for generating the output. The goal of the encoder 202 is to transform the input ECG data signal into a compressed representation while retaining as much useful information as possible and disregarding redundant features. The decoder 204 generates a reconstructed version of the input ECG data signal based on the latent representation.

The quality of the output of the decoder 204 relies on the effectiveness of the encoder 202 in generating a meaningful latent representation. Therefore, parameters of the encoder 202 and decoder 204 are optimized to reduce error between the input ECG data signal and the corresponding reconstructed output. The latent representation is further optimized by the attention layer 210 to generate weighted representations. The MLP classifier 220 is optimized to perform binary classification of pain threshold and tolerance. Other types and arrangements of classifiers can be used in addition to or in place of the MLP classifier 220.

As indicated above, the attention layer 210 is used to generate a weighted representation in accordance with the latent representation. It takes the latent representation and produces weights. The weights are then used to weight the contributions of corresponding input elements to the output of the decoder 204. The attention layer 210 illustratively comprises a hyperbolic tangent (tanh) layer which is a non-linear activation function that is used to normalize the input of the attention layer 210. The attention layer 210 further comprises a softmax layer that takes as its input the latent representation and produces a weight score used to weight the contributions of the input elements to the output of the decoder 204. A final weighted representation is generated by the attention layer 210 as a weighted sum of the latent representation.

The parameters of the encoder 202 and decoder 204 are trained to minimize the reconstruction error between the original input ECG data signal and the reconstructed output ECG data signal of the decoder 204, illustratively using a mean squared error function, although other types of error functions can be used. The weighted representation and one or more corresponding labels are used to optimize the MLP classifier 220, illustratively by training using a categorical cross-entropy loss function.

In some embodiments, features such as heart rate variability (HRV), inter-beat interval

(IBI), statistical features (SF), and heart rate (HR) features, are extracted from the input ECG data signal. The latent representation is learned from the input ECG data signal by interaction of the encoder 202 and the decoder 204 in the DADAED neural network 200. The latent representation is further optimized by the attention layer 210 to generate weighted representations, and the MLP classifier 220 is optimized to perform binary classification of pain threshold and tolerance, as will now be described in more detail.

For a given input ECG data signal X, a noisy input signal X̅ is first generated according to the original signal X. The noisy signal is then fed into the encoder 202. The encoder 202 implements an encoder function f_{θ} and generate a latent representation r:

$$r = {f}_{θ} ( \overline{X} ) \qquad ( 1 )$$

The latent representation r is then fed into the decoder 204. The decoder 204 implements a decoder function g_{ϕ} and generates an output X̅′:

$${\overline{X}}^{′} = {g}_{ϕ} ( r ) \qquad ( 2 )$$

As indicated previously, the attention layer 210 is used to generate weighted representation according to the latent representation r. It takes as its input the latent representation of the ECG data signal and produces a weight score. The weights are then used to weight the contribution of the input element to the output of the decoder 204. The attention layer 210 includes a tanh layer and a softmax layer, as described above. The attention layer 210 implements the following computations:

$$e = tanh ( W h + b ) \qquad ( 3 )$$

$$w = softmax ( e ) \qquad ( 4 )$$

where W is a weight matrix, b is a bias term, tanh is the hyperbolic tangent function, softmax is the softmax function, e is the weight score, and w is the attention weigh. A final weighted representation h is generated at the output of the attention layer 210 as a weighted sum of latent representation r:

$$h = \sum w ⊙ r \qquad ( 5 )$$

The parameters of the DADAED neural network 200 are trained to minimize the construction error between the original ECG input X and the reconstructed output X̅ of the decoder 204, illustratively using a mean squared error function:

$$E = \frac{1}{N} \sum {‖ X - {\overline{X}}^{′} ‖}_{2}^{2} + λ {‖ W ‖}_{2}^{2} \qquad ( 6 )$$

where N is the total number of trainable samples of the input ECG data signal X, and λ∥Wλ2/2 denotes the regularization term in the latent representation layer. The weighted latent representation and the label y are used to optimize the MLP classifier 220 which implements MLP model f_{Ψ}. In illustrative embodiments, the MLP classifier 220 includes a plurality of neural network layers that are trained using a categorical cross entropy loss function:

$${L}_{C} = - \frac{1}{N} {\sum }^{N} {\sum }^{C} y log ( \overline{y} ) \qquad ( 7 )$$

where C is the number of classes in the classification problem, y is the true label, and y̅ is the predicted probability of the output of the MLP classifier 220. The parameters of the full DADAED neural network 200 are then optimized by minimizing the objective function:

$$L = \sum α E + {α}_{C} {L}_{C} \qquad ( 8 )$$

where α and α_{c} are regularization weights assigned to the respective error functions E and L_{c} of Equations (6) and (7).

FIG. 3 shows another example of a DADAED neural network 300 in an illustrative embodiment. In this embodiment, the DADAED neural network 300 illustratively comprises two encoder-decoder networks, namely, encoder 302-1 paired with decoder 304-1 and encoder 302-2 paired with decoder 304-2. The DADAED neural network 300 further comprises an attention layer 310 and an MLP classifier 320. The first one of the encoder-decoder networks comprising encoder 302-1 and decoder 304-1 is driven by a noisy version of an input PPG data signal X₁, and the second one of the encoder-decoder networks comprising encoder 302-2 and decoder 304-2 is driven by a noisy version of an input ECG data signal X₂, with each encoder-decoder pair producing a separate latent representation that is processed by the attention layer 310 and the MLP classifier 320 in a manner similar to that previously described in conjunction with the illustrative embodiment of FIG. 2. The index j in this embodiment illustratively denotes, for example, a j-th training sample.

It is to be appreciated that the particular neural network arrangements illustrated in FIGS. 2 and 3 are examples only, and other types of denoising encoder-decoder neural networks can be used in other embodiments, configured to process additional or alternative input data signals, and to generate a wide variety of different types of classification outputs. For example, additional input data signals can be accommodated by increasing the number of encoder-decoder pairs in the DADAED neural network.

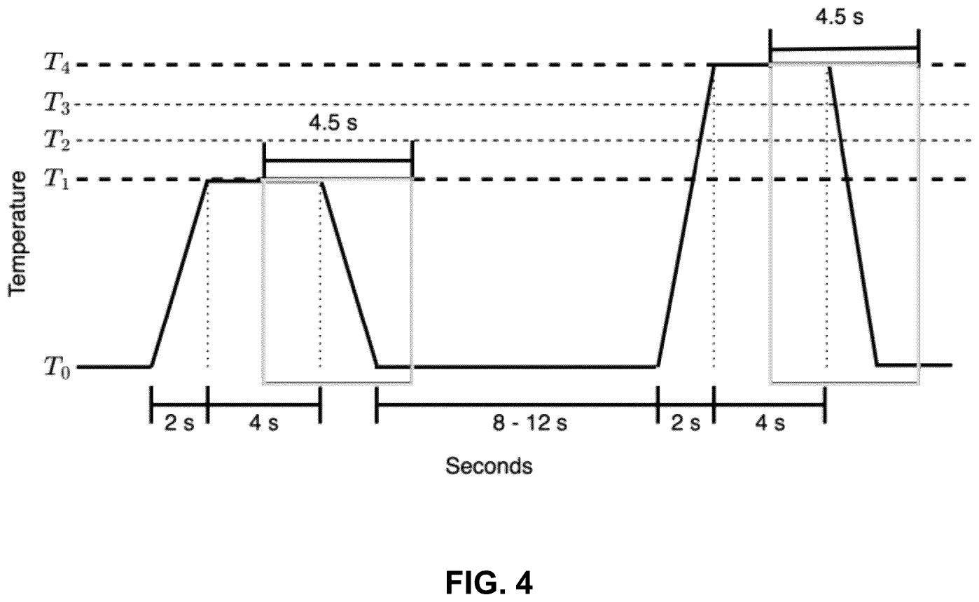

Results of experiments performed on an example implementation of the illustrative embodiment of FIG. 2 will now be described with reference to FIGS. 4 through 6.

In these experiments, the performance of the DADAED neural network 200 of FIG. 2 for pain recognition is evaluated using the BioVid Heat Pain Database, which includes data from a previous pain threshold and pain tolerance study involving a total of 90 healthy subjects (45 male and 45 female), as described in S. Walter et al., “The BioVid Heat Pain Database,” IEEE International Conference on Cybernetics, pp. 128-131, 2013, which is incorporated by reference herein in its entirety. The subjects were between 18 and 65 years old. Each subject was assessed prior to the study to identify and exclude preexisting neurological, chronic pain, cardio-vascular disease, a daily pain medication user and other pain related conditions. The study was conducted in accordance with ethical guidelines set out in the WMA Declaration of Helsinki and all subjects received expense allowance. The database includes data signals from ECG, EMG and GSR sensors, and corresponding video recordings. These modalities were recorded concurrently during the study. A thermode was used for the pain elicitation. Four temperatures for stimulation were equally distributed between pain threshold and pain tolerance of the subjects. The intensity was calibrated by dividing the range between when pain is felt (threshold) and when pain is unbearable (tolerance) for each participant.

FIG. 4 illustrates an example signal segmentation and pain quantification configuration, plotting applied temperature of a pain stimulus as a function of time in seconds. This example configuration is utilized in illustrative embodiments of the present disclosure, but is only an example, and numerous other configurations can be used in other embodiments.

As shown in FIG. 4, each stimulation of the example configuration comprises a ramp-up phase of about 2 seconds, a peak stimulation phase of about 4 seconds, and then a ramp-down phase of about 2 seconds, and is immediately followed by a recovery phase with a random duration in the range of about 8 to 12 seconds. A baseline temperature T₀ (32° C., 89.6° F.) corresponds to the temperature applied during the recovery phase. Each of the five stimulation levels T₀, T₁, T₂, T₃, T₄ of heat-induced pain was randomly stimulated a total of 20 times to give a total of 80 responses, thus, 20×5=100 samples. Therefore, the raw signal includes 90×100=9000 samples. The signals are labelled accordingly with its corresponding heat-stimulated pain level (T₀, T₁, T₂, T₃, T₄).

For the experiments performed on the example implementation of the FIG. 2 embodiment, preprocessing operations were initially performed as follows. As the BioVid Heat Pain sensor data was collected using a sampling rate of 512 Hz, the sampling rate of the ECG data signal was downsampled to 128 Hz to reduce the computational requirements. Also, band-pass filtering was applied to remove significant noise and artifacts from the ECG data signal. The resulting filtered signals are further segmented into a 4.5 second window length with a shift of 4 seconds from the stimulation onset at the beginning of the ramp-up, as illustrated in FIG. 4. Each ECG bio-signal within a given window constitutes a one-dimensional array of size 4.5×128=576. The dimension of each tensor for a single subject is therefore 4.5×576×1. The signal went through augmentation on the above-noted windows of length 4.5 seconds with a temporal shift of 4 seconds from the stimulation onset.

In the experiments, in addition to the DADAED features, the above-noted HRV, SF, IBI, and HR features were systematically extracted, in order to help improve the accuracy and robustness of the MLP classifier 220. In some embodiments, the DADAED features and the additional extracted features are illustratively combined in a late fusion manner to classify pain threshold and pain tolerance in the MLP classifier 220. The deep neural network layers of the MLP classifier 220 illustratively utilize a Rectified Linear Unit (ReLU) activation function:

$${ReLU}_{α} ( x ) = max ( 0 , x ) \qquad ( 9 )$$

The ReLU activation function prevents the vanishing gradient problem and accelerates training of the MLP classifier 220, although other activation functions can be used in other embodiments. For the output layer of the MLP classifier 220, a softmax activation function was used to normalize the output and provide probabilities for the binary classification of pain threshold and tolerance. An Adam optimizer was used to train all architectural structures with a learning rate of 0.001. The training process is set at 100 epoch cycles with a batch size of 32, and the Gaussian noise factor parameter is set to 0.2. The training was implemented using keras, biospy, scikit-learn, and tensorflow libraries.

To evaluate the model performance, leave-one-out-cross-validation (LOOCV) was used on the 90 subjects. This means that of the 90 subjects, LOOCV would involve training the model on 89 subjects and testing it on the one remaining subject. This process is repeated 90 times with each subject being left out in turn, and the performance averaged over all 90 runs. LOOCV provides an unbiased evaluation of the model performance, as it uses all available samples for training and testing.

In evaluating the effectiveness of the above-described experimental arrangement for pain recognition, evaluation metrics including accuracy, F1 score, and ROC AUC score were used for the classification task, where ROC denotes Receiver Operating Characteristic and AUC denotes Area Under the Curve, with the curve more particularly being the ROC curve. The sample set comprises five classes (baseline stimulus T₀, threshold stimulus T₁, two intermediate stimuli T₂, T₃, and tolerance stimulus T₄). The objective of the classification task in illustrative embodiments is two-fold: 1) discriminate between baseline T₀ and pain threshold T₁; and 2) discriminate between pain threshold T₁ and pain tolerance T₄. Accordingly, two distinct binary classification tasks were performed in the MLP classifier 220.

Table 1 below shows the classification results in terms of accuracy and F1 score for the experiments, in discriminating baseline temperature versus pain threshold and pain threshold versus pain tolerance. Accuracy and F1 score results are initially shown for additional extracted features, referred to herein as “hand-crafted” or HC features, illustratively comprising the above-noted HRV, SF, IBI and HR features, then for the features automatically extracted into the latent representation by the DADAED neural network, and finally for the combined feature sets including the latent representation of the DADAED neural network plus the HC features. The results show that the DADAED neural network achieved an accuracy and F1 score of about 85% for pain threshold and about 72% for pain tolerance. Additional improvements are achieved in the illustrative embodiment in which the DADAED neural network latent representation is supplemented with the HC features.

TABLE 1

| | T₀ vs. T₁ | | T₁ vs. T₄ | |
|---|---|---|---|---|
| Features | Accuracy | F1 score | Accuracy | F1 score |
| HC | 0.768 | 0.768 | 0.664 | 0.664 |
| DADAED | 0.853 | 0.853 | 0.716 | 0.716 |
| DADAED + HC | 0.862 | 0.862 | 0.725 | 0.725 |

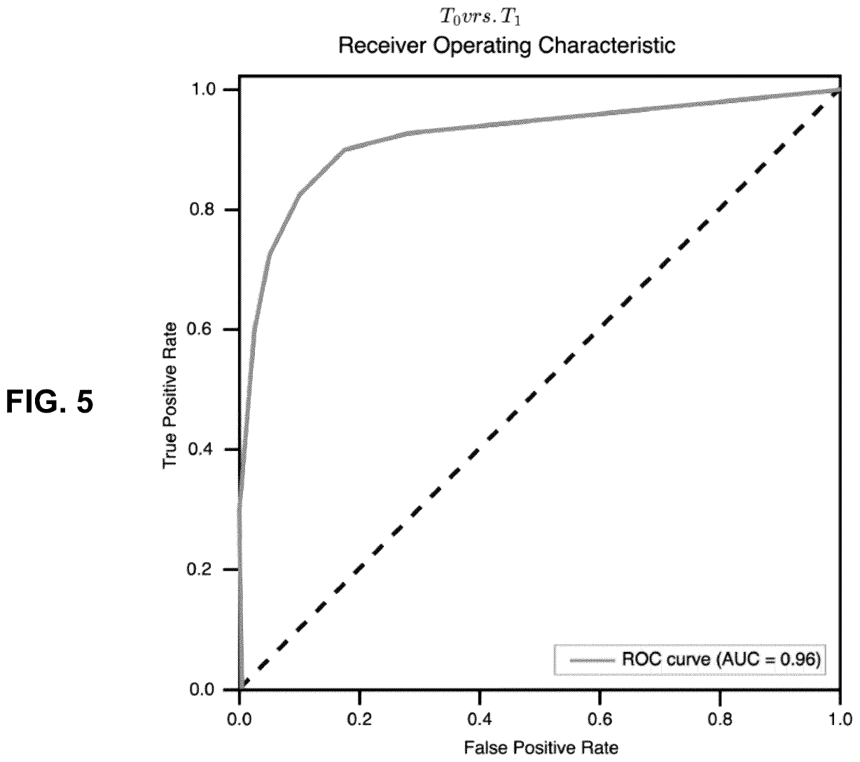

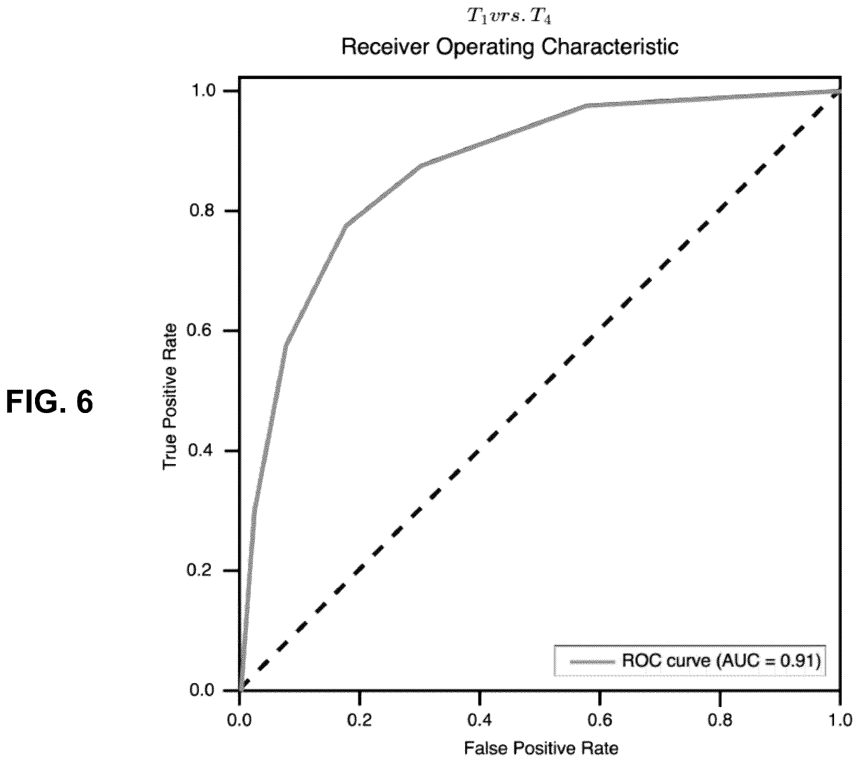

FIGS. 5 and 6 show the performance results in terms of ROC AUC score for the respective two classification tasks identified above, namely T₀ vs. T₁ and T₁ vs. T₄. These figures plot the ROC curves for the respective classification tasks, and provide the corresponding AUC scores for the ROC curves. The ROC curves show the corresponding classification performance as the discrimination threshold of the MLP classifier 220 is varied.

The ROC curves indicate that the MLP classifier 220 exhibits good performance in distinguishing pain threshold and pain tolerance, with ROC AUC scores of 0.96 for the ROC curve of FIG. 5 corresponding to the T₀ vs. T₁ classification, and 0.91 for the ROC curve of FIG. 6 corresponding to the T₁ vs. T₄ classification.

The experimental results described above demonstrate that illustrative embodiments provide significant advantages in terms of accuracy in discriminating pain threshold and pain tolerance over conventional approaches. For example, the illustrative embodiments in these experiments achieved accuracy of 86% and 73% in the DADAED+HC case, using only ECG data signals.

Additional experimental results were obtained utilizing k-fold cross validation, with k=10, to further illustrate the effectiveness of the example machine learning model used in the DADAED neural network 200.

It was found that the model can generalize well across unseen data. More particularly, testing accuracy (accuracy on unseen data) is consistently better than training accuracy in illustrative embodiments. This indicates that the model in such embodiments has a strong generalization capability, as it indicates that the model has learned to capture underlying patterns in the data rather than memorizing the training examples.

Individual differences across a dataset refer to the variability or diversity in the data points within the dataset. In the context of machine learning and data analysis, the term relates to the fact that different data points (or individuals) in the dataset may exhibit unique characteristics, behaviors or patterns. These individual differences can be due to various factors including inherent variability in the data, individual-specific traits, relationship with pain as far as this data is concerned, or environmental factors.

For example, in a dataset with data involving multiple human subjects, individual differences could manifest as variations in responses to a stimulus, differences in physical characteristics, variations in preferences, or variations in performance on a task. These differences are often what make datasets interesting and reflect the complexity of the real-world phenomena being studied.

In the above-noted k-fold cross validation, with k=10, it was found that the average individual difference for the pain threshold is 0.0004655. This value represents the difference in performance metrics (e.g., accuracy) between the model's performance on a single fold of the k-fold cross validation and the average performance across all folds of the k-fold cross validation. It quantifies how much the model's performance varies from one fold to another.

The magnitude of the individual difference is also quite small. Smaller values indicate that the model's performance is relatively consistent across different subsets of the data. In other words, the performance of the model does not vary significantly from one fold to another.

The individual difference value noted above suggests that, on average, the model's accuracy on a single fold is very close to the average accuracy across all folds. In practical terms, it means that the model generalizes well across different subsets of the data and is relatively robust.

For the pain tolerance, the average individual difference is 0.0023988. Again, a small individual difference value such as this is a positive sign as it indicates that the model's performance is stable and consistent across different subsets of the data.

As described above, the DADAED neural network 200 of FIG. 2 is illustratively configured to automatically detect pain threshold and pain tolerance, and can be deployed in a wide variety of different use cases to help improve the quality of care for patients with pain-related conditions and contribute to advancements in pain management and research. Numerous alternative embodiments are possible. For example, as previously described in conjunction with FIG. 3, the DADAED neural network 300 is further configured to process an input PPG data signal from one or more PPG sensors. These and other embodiments can predict pain levels in real time or near real time. A given DADAED neural network of the type disclosed herein can also be incorporated into a wide variety of different pain biomarker technologies. These and numerous other illustrative embodiments will be further described below.

As indicated previously, illustrative embodiments disclosed herein provide significant improvements over conventional approaches to pain recognition and in numerous other diagnostic applications.

For example, some embodiments disclosed herein provide automated machine learning and/or AI-based generation of a pain biomarker as an important additional diagnostic vital sign for patients and other individuals.

In some embodiments, accurate and automatic pain recognition is provided utilizing low resolution electrical and light-based cardiac sensors to improve pain monitoring and treatment. For example, a DADAED neural network as disclosed herein processes non-invasive physiological data to objectively describe pain threshold and tolerance which can further be transferred to solving issues relating to emotional distress.

Illustrative embodiments disclosed herein include closed-loop systems with AI-based pain estimation functionality for effective pain management, although the disclosed techniques can be implemented in numerous other types of systems and devices.

As additional examples, some embodiments can be configured to automatically control wearable devices and/or internal devices for pain relief. Additionally or alternatively, some embodiments can be configured to trigger ordering and/or delivery of pre-approved medications to a patient.

Other examples include military applications in the field, in which rapid detection, assessment and diagnosis of pain and related injuries is particularly important.

Still further examples include embodiments that are configured to provide pain indicators to other systems or devices for further processing.

These and other examples set forth herein are illustrative of automated actions that can be undertaken under the control of a DADAED neural network or other machine learning system as disclosed herein.

Illustrative embodiments disclosed herein meet the need for more accurate and reliable pain biomarkers that can be used in a wide range of clinical settings. Such embodiments implement improved deep learning approaches that are configured to extract features relating to pain in addition to other features such as heart rate variability, inter beat interval, heart rate and/or statistical features.

The deep learning model in some embodiments is implemented as a DADAED neural network that captures at least one latent representation that can more accurately detect and discriminate pain threshold and pain tolerance.

Some embodiments also employ one or more attention layers that enable the deep learning model to focus on specific parts of the input that are more relevant in real-world applications when making predictions.

Additionally or alternatively, some embodiments are configured to take into consideration knowledge about emotions, age, and other individual differences for more optimal performance on pain discrimination.

Illustrative embodiments recognize that pain is a complex multifaceted experience that involves sensory, affective, and cognitive dimensions. Accordingly, some embodiments focus on lower-resolution ECG and/or PPG data to gain deeper understanding of the specific aspects of the pain that it captures, unlike other approaches that use too many modalities and therefore become overwhelming and difficult to interpret.

In some embodiments, generation of an AI-based pain biomarker to predict pain threshold and tolerance in order to facilitate effective pain management and treatment illustratively involves the following steps, although it is to be appreciated that additional or alternative steps, possibly applied in a different order, can be used in other embodiments.

1. Identify one or more input signals and associated pain biomarkers. This step identifies one or more physiological and/or behavioral signals that are associated with pain. In some embodiments, ECG and/or PPG data signals are identified as such data signals can measure electrical activities of the heart, provide information about changes in heart rate and rhythm in response to pain, measure changes in blood flow and volume in the skin, and/or provide information about changes in peripheral circulation in response to pain.

2. Collect and annotate data. The ECG and/or PPG data signals are illustratively collected from wearable devices of respective patients or other individuals. The data is annotated with the relevant pain biomarkers, such as baseline, pain threshold, pain intensity, and emotional level.

3. Train the deep learning model. With the annotated data, the deep learning model can be trained to recognize patterns and associations between the biomarkers and pain. The model parameters are optimized to achieve high accuracy and generalization.

4. Validate the deep learning model. After training the model, it is validated using independent new datasets and metrics. This ensures that the model is reliable and generalizes well to new cases.

5. Integrate the deep learning model. Once the model is validated, it is integrated into a clinical or research setting. This illustratively involves providing an intuitive user interface for healthcare providers and patients, and ensuring that the performance of the model is consistent across different environments and patient populations.

6. Continuously improve the deep learning model. Finally, the model is continuously monitored and improved over time as more data becomes available and new pain biomarkers are discovered.

Some embodiments implement these or other steps in implementing a deep learning-based approach to understanding pain features and providing an AI-based pain biomarker to predict pain threshold and tolerance and emotional distress.

These and other embodiments can advantageously provide improved pain assessment, personalized treatment plans, and better patient outcomes. For example, the disclosed AI-based pain biomarker arrangements can transform the field of pain research and management, by providing objective and personalized measures of pain that can inform diagnosis, treatment, and drug development.

As indicated previously, some embodiments focus directly on ECG and/or PPG data signals in lower dimensions. This illustratively involves utilizing downsampled or otherwise lower dimension ECG and/or PPG data signals. The lower dimension ECG and/or PPG data signals are an example of what is more generally referred to herein as a “noisy version” of a corresponding higher dimension ECG and/or PPG data signal. Accordingly, generating a “noisy version” of a given input data signal can involve, for example, downsampling the input data signal or otherwise reducing the dimensionality of the input data signal. The term “noisy version” as used herein is therefore intended to be broadly construed. Lower dimensional ECG and/or PPG data signals can be processed more efficiently than corresponding higher dimensional data signals. This advantageously results in significantly faster training and inference times for the deep learning model.

In some embodiments, the deep learning model first introduces noise to the input ECG and/or PPG data signals to help improve generalization ability by making it more robust to variations and noise in the input data. This helps in the training phase as the model learns to capture the underlying patterns and features in the data, rather than memorizing the noise or idiosyncrasies of the training data.

Additionally or alternatively, denoising functionality is implemented in the encoder-decoder neural network. Such denoising functionality makes the input data more consistent and easier to process, which improves the accuracy and efficiency of the model in illustrative embodiments.

Some embodiments also implement one or more attention layers in the encoder-decoder neural network, for example, to allow the model to focus on specific parts of the input information when making predictions, resulting in improved performance.

In some embodiments, an AI-based pain biomarker is configured to take into account individual differences and emotional states to personalize treatment. This is important to provide personalized pain management strategies that are tailored to the unique needs of individual patients. Knowledge about individuals helps reduce bias in pain diagnosis and treatment which has shown to disproportionally impact certain demographics groups. The goal is also to improve each patient's outcome. By providing personalized pain management strategies, the AI-based pain biomarker can improve each patient's outcome and quality of life. Incorporating individual differences in data used to train the model also captures the complexity of pain recognition and produces more accurate predictions.

As indicated previously, pain is a complex and multifaceted experience that involves sensory, affective, and cognitive dimensions. Illustrative embodiments herein focus on low sampling resolution ECG and/or PPG data to maximize accuracy and understanding of the specific aspects of pain captured by the data. Such arrangements better characterize the ANS response to pain in the ECG and/or PPG data, using one or more attention layers to better focus on the specific information/features that correctly correlate to pain detection, leading to significantly improved results.

In some embodiments, lower dimensional input data is utilized to improve performance of the deep learning model.

For example, some embodiments use a combination of sensors of different types for pain assessment, such as the above-noted lower dimensional ECG and/or PPG data, possibly in combination with one or more other data types, such as video, facial electromyography (EMG), blood pressure, etc. The disclosed embodiments can also provide excellent classification results using signals from a single source.

In some embodiments, lower dimensional data is obtained from a wearable device or other medical monitoring device, such as a Kardiamobile device. This can include single-lead sensor devices that serve as a single source of ECG data, PPG data or another type of data. Thus, instead of utilizing a full-dimension 12-lead ECG data signal providing 12-dimensional data as a separate signal for each lead, illustrative embodiments utilize lower dimensional data, such as a single-lead ECG signal from a medical monitoring device.

In some embodiments, lower dimensional representations are utilized to reduce data complexity, remove noise, and highlight important patterns in the data, and also to decrease the computational burden and hardware requirements for real time analysis of pain.

In some embodiments, lower resolution refers to the level of detail or coarseness in the data. For example, the lower resolution may refer to a lower sampling rate or fewer data points per unit of time. Thus, illustrative embodiments can be configured to use a significantly reduced sampling rate of 128 Hz or less, rather than the 512 Hz to 1 KHz sampling rates typical of clinical ECG systems. This again reduces computational burden and memory requirements for real time analysis of pain. Such reductions in sampling rate can be viewed as illustratively generating a “noisy version” of a higher resolution data signal in some embodiments, as that term is broadly used herein.

Similarly, PPG sensors that capture data from a single source, such as a fingertip or earlobe, use optical signals to track pulse and typically have low resolution via a low sampling rate. As indicated above, illustrative embodiments utilize PPG data, possibly in combination with ECG data and/or other types of data, for accurate pain discrimination. Again, generating a noisy version of such a signal can comprise, for example, downsampling the signal or otherwise reducing the sampling rate and/or dimensionality of the signal.

Illustrative embodiments of the denoising encoder-decoder neural network can be configured to operate with ECG data alone, PPG data alone, a combination of ECG and PPG data, or other combinations of ECG and/or PPG with other types of data. PPG can provide additional information beyond pulse rate, such as pulse amplitude modulation, and is a reflection of blood flow and vasculature, while ECG generally assesses electrical conductances. A combined ECG/PPG system may be configured as a two-dimensional (e.g., two sensor), low sampling rate system for ease of administration and reduced computational requirements, although numerous other arrangements are possible.

Other embodiments can utilize numerous alternative arrangements of lower dimensional sensors, including, for example, single lead/electrode, 2 leads/electrodes, 3 leads/electrodes, etc. Such arrangements are lower dimensional relative to, for example, the above-noted full-dimension 12-lead ECG arrangement.

As mentioned above, noise is introduced in some embodiments by reducing the dimensionality of the input data, illustratively by, for example, reducing the amount and/or type of data in the data signal, downsampling or otherwise reducing the sampling rate, or using other techniques, all of which are considered to result in what is more generally referred to herein as a “noisy version” of a higher dimensional data signal. In some embodiments, the noisy version is generated directly by one or more sensors through internal processing of those sensors that results in dimensionality reduction.

In some embodiments, electroencephalography (EEG) data signals that measure brain activity associated with pain can be used, in addition to or in place of the above-described ECG and/or PPG data signals. Such an embodiment is configured to use machine learning to analyze patterns of brain activity and identify those associated with pain.

Another alternative version of an AI-based pain biomarker in illustrative embodiments herein is implemented in a system that uses machine learning to analyze changes in a person's voice to detect pain. Such a system is illustratively configured to analyze changes in pitch, tone, and other vocal characteristics that are associated with pain, and use that information to provide a pain score.

Additionally or alternatively, a system as disclosed herein can be configured to implement an AI-based pain biomarker by using computer vision to analyze changes in a person's facial expression to detect pain. Such a system is illustratively configured to use algorithms to track changes in facial muscles and expressions that are associated with pain and use that information to provide a pain score.

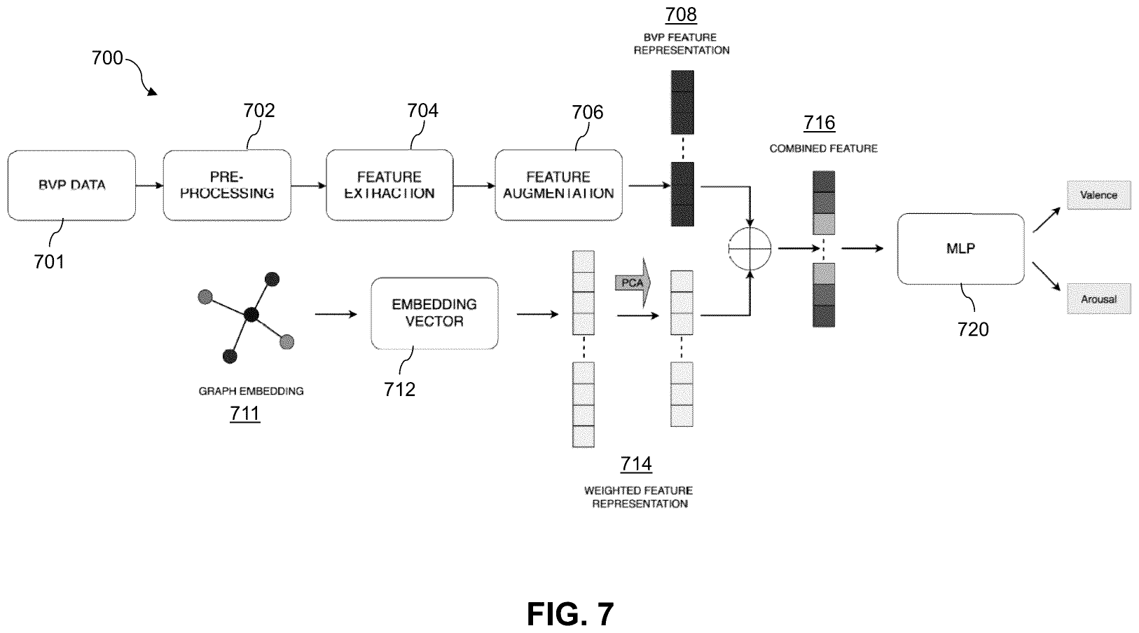

Additional illustrative embodiments will now be described with reference to FIG. 7. These embodiments provide deep learning based emotion recognition using input data signals from one or more fingertip blood volume pulse (BVP) sensors, which are illustratively a type of PPG sensor as that term is broadly used herein. Such embodiments can be utilized in conjunction with the DADAED neural networks previously described in conjunction with FIGS. 1 through 6. For example, an MLP classifier of the type described previously can be configured to process additional features extracted from a BVP data signal. An MLP classifier configured to process such features will be described in greater detail below. Such features, as well as additional or alternative features in any combination, can be provided as additional extracted feature inputs to the MLP classifiers 220 and 320 of FIGS. 2 and 3 in some embodiments, in a manner similar to that previously described for the HC features.

Emotions are central to physical and mental health and general wellbeing. There is a great need to affordably and non-invasively track moment-to-moment changes in emotional states and their association with chronic conditions. Illustrative embodiments utilize data from BVP sensors. A BVP sensor is a widely used sensor for measuring blood volume changes and heart rate, and is embedded in numerous biofeedback systems and applications. For example, the BVP sensor may be in a non-invasive form factor, such as form factor configured for attachment to a fingertip of a subject. Some of the disclosed embodiments utilize features extracted from the BVP sensor, such as heart rate variability (HRV), multi-scale entropy (MSE), power spectral density (PSD), IBI, tachogram power and/or statistical moments (e.g., mean, standard deviation, skewness and kurtosis), as derived from BVP data signals. Such features in some embodiments are weighted by additional features such as age and gender of participants. As will be described, an MLP classifier trained to perform classification tasks for emotion recognition using such features exhibits significant improvements in accuracy and FI score in valence and arousal dimensions.

These and other embodiments can be incorporated into closed-loop emotion detection and intervention systems, and in numerous other types of systems and devices. Such arrangements can increase emotional awareness and regulation to ultimately alleviate suffering. For example, illustrative embodiments implement machine learning models within a preventive perspective to minimize the risk of developing chronic conditions and to enhance health and wellbeing. Numerous other arrangements are possible.

In some embodiments, the above-noted features such as MSE, PSD, IBI, tachogram power, and/or statistical moments, in addition to one or more HRV features, are extracted from one or more BVP data signals. Original features and normalized features are then augmented to capture non-linearity and learn information specific instances dealing with time-varying drift. Participant age and gender may be embedded as weights to information-specific augmented features. As will be described, such arrangements can provide significantly better recognition accuracy than conventional approaches.

FIG. 7 shows an example machine learning system 700 configured to utilize the above-described features for valence-arousal emotion state classification. The machine learning system 700 processes input BVP data 701. The BVP data 701 is generally indicative of the volume of blood that passes through tissues in a particular area with each pulse of the heart, and is illustratively collected from a fingertip BVP sensor. As indicated above, such a BVP sensor may be viewed as an example of a PPG sensor, and is illustratively configured to measure variations in blood volume in the capillaries and arteries relating to variations in blood flow from which heart rate and other more complex features can be derived. After data collection, the BVP data 701 is subject to preprocessing 702 to remove non-physiological noise. Then, relevant features of the type previously described are extracted via feature extraction 704.

After feature extraction 704, significant variations may be observed between the extracted features for different participants. This is because different participants respond differently to the same stimulus. Accordingly, the machine learning system 700 is configured to implement feature augmentation 706 in generating a BVP feature representation 708. This initially involves using min-max normalization to scale the original features to a 0-1 scale:

$${x}^{′} = \frac{x - min ( x )}{max ( x ) - min ( x )} \qquad ( 10 )$$

where x is the original feature and x′ is the normalized feature. Given an original feature x and normalized feature x′, the corresponding augmented feature x is represented by:

$$\overline{x} = [ \begin{matrix}x \\ {x}^{′}\end{matrix} ] \qquad ( 11 )$$

Such augmented features capture nonlinearity and learn information-specific instances dealing with time-varying drift. These augmented features are also denoted in further description below as BVP features B.

The machine learning system 700 is further configured to utilize a graph embedding 711. The graph embedding 711 learns a feature representation for each node of a given network in low dimension. In illustrative embodiments, the graph embedding 711 represents the age and gender of participants and is used to weight the information-specific augmented features described above. Graphs illustratively comprise directed graphs represented as triples. A corresponding graph embedding is illustratively defined as:

$$G = ℰ \times R \times ℰ \qquad ( 12 )$$

where ε is a set of entities and R is a set of relations. Random binary variables {0,1} indicate whether a triple exists. To increase the possibility of existing triples, latent graph features are extracted. These graph features are denoted as weighted features G and are represented in the form of an embedding vector 712.

The augmented features and the graph features are combined in the machine learning system, as will now be described in more detail. As indicated in the figure, a weighted feature representation 714 is generated from the embedding vector 712. This illustratively involves applying principal component analysis (PCA) to reduce the dimension of the graph features to match that of the augmented features, where G̅ is the representation of the graph features after dimensionality reduction. A transmutation matrix T is then used to configure the graph features for weighting of the augmented features, with the combined feature 716 being given by a combined feature vector C:

$$C = B ⊕ ( T \overline{G} ) \qquad ( 13 )$$

where ⊕ indicates the direct sum. The combined feature vector C is applied as an input to a deep neural network of an MLP classifier 720.

The MLP classifier 720 illustratively comprises four hidden layers and an output layer. After each hidden layer, batch normalization and ReLU activation is applied. Dropout is set at 0.5. An Adam optimizer and a mean square error (MSE) loss function is utilized for training of the MLP classifier 720. The MLP classifier 720 utilizes a softmax activation function for final valence-arousal classification.

It is to be appreciated that the components and configuration of the machine learning system 700 are presented by way of illustrative example only, and additional or alternative components arranged in different configurations can be used in other embodiments.

Experiments were performed on an example implementation of the machine learning system 700, utilizing the DEAP database, which contains data collected in a study of emotion analysis using physiological signals, as described in S. Koelstra et al., “DEAP: A Database for Emotion Analysis Using Physiological Signals,” IEEE Transactions on Affective Computing, Vol. 3, No. 1, pp. 18-31, 2012, which is incorporated by reference herein in its entirety. The DEAP database was recorded purposely for the analysis of human affective states. Thirty two subjects participated in the study, fifty percent of which were male and fifty percent of which were female. Their ages ranged between 19 and 37 years, with a mean age of 26.9.

Before the study, each participant signed a consent form and filled out a set of questionnaires. Participants were also instructed to read information detailing the experimental protocol instructions and the meaning of different scales used for self-assessment. All questions regarding the protocol were answered by a study leader. Each participant was escorted to a laboratory once all instructions were clear. The participants then watched 40 1-minute long excerpts of music video and had their physiological signals synchronously collected. After watching each video, participants were asked to rate them in terms of their level of arousal and valence, on a continuous scale of 1-9 for each of arousal and valence. A BVP sensor was used to measure blood volume in the thumb of each participant.

The BVP sensor data was used to compute the local maxima, IBI, and HRV features. The BVP signal was recorded at 512 Hz. The preprocessing 702 illustratively included downsampling the data to 128 Hz, removing noise and artifacts and implementing a low bandpass frequency filter. Self-assessment ratings on the valence and arousal dimensions were used for emotion recognition.

Feature extraction 704 illustratively involved extracting features from the BVP data signal for the entire 60 second duration of the data collection for each video. Accordingly, there were 1280 trials, also referred to as samples, corresponding to 32 subjects×40 videos, that were investigated to assess participant emotions. The features extracted from the BVP signals of the respective samples illustratively included HRV, IBI, MSE, tachogram power, PSD and statistical moments.

Valence and arousal were defined as two binary classification tasks in the experiments. Accuracy and F1 score metrics are used to evaluate results of the MLP classifier 720 in performing these classification tasks. The F1 score takes class balance into consideration, contrary to the accuracy which simply reports classification rates. To deter individual differences from affecting classification performance, the final combined feature vectors were normalized using Equation (1) above. Then, for each of the 32 participants, to evaluate the performance of emotion classification in an LOOCV approach, the accuracy and F1 score metrics were computed. In accordance with the LOOCV approach, at each step of the cross validation, the data of one participant is used as test data and the rest of the data is used as training data. This is done 32 times, with a different participant left out each time.

Table 2 below shows the average accuracies and F1 scores over participants for each BVP feature, the combination of BVP and graph embedding features and their rating scale within the valence-arousal dimension.

TABLE 2

| | Valence | | Arousal | |
|---|---|---|---|---|
| Features | Accuracy | F1 score | Accuracy | F1 score |
| BVP | 78.8 | 75.7 | 77.2 | 73.5 |
| BVP + GE | 85.3 | 84.1 | 81.6 | 80.8 |

The results show a classification accuracy of 85.3% in the valence dimension and 81.6% in the arousal dimension. Similarly, classification F1 scores were 84.1% in the valence dimension and 80.8% in the arousal dimension. These measures are for the case of classification of the combined BVP and graph features.

The results further indicate that classification of the combined BVP features and graph features fares significantly better than only the BVP features alone by 6.5% and 8.4% in terms of accuracy and F1 score respectively in the valence dimension and 5.9% and 7.3% for accuracy and F1 score respectively in the arousal dimension.

It was also generally found that the results for both BVP features alone and BVP features plus graph features provided significant improvements relative to conventional approaches.

As indicated above, various features and functionality of the FIG. 7 embodiment can be implemented within or otherwise in combination or in conjunction with one or more of the embodiments described in conjunction with FIGS. 1 through 6. For example, a given embodiment can incorporate various aspects of the graph embedding and weighted feature aspects of machine learning system 700 into the neural network 110 of the FIG. 1 system, or into the neural networks 200 and 300 of FIGS. 2 and 3.

Again, the particular neural networks and their components and associated input signals as described above are considered illustrative examples only.

It is to be appreciated that the particular use cases described herein are examples only, intended to demonstrate utility of illustrative embodiments, and should not be viewed as limiting in any way.

Automated remedial actions taken based on outputs generated by one or more neural networks and an associated AI-based classification algorithm of the type disclosed herein can include particular actions involving interaction between a processing platform implementing the classification algorithm and other related equipment utilized in one or more of the use cases described above. For example, outputs generated by an AI-based classification algorithm as disclosed herein can control one or more components of a related system. In some embodiments, the classification algorithm and the related equipment are implemented on the same processing platform, which may comprise a computer, a smartphone, a wearable device, an internal device, an intelligent medical monitoring device, a handheld sensor device or other type of processing device.

It should also be understood that the particular arrangements shown and described in conjunction with FIGS. 1 through 7 are presented by way of illustrative example only, and numerous alternative embodiments are possible. The various embodiments disclosed herein should therefore not be construed as limiting in any way. Numerous alternative arrangements of neural networks and associated AI-based classification algorithms can be utilized in other embodiments. Those skilled in the art will also recognize that alternative processing operations and associated system entity configurations can be used in other embodiments.

It is therefore possible that other embodiments may include additional or alternative system elements, relative to the entities of the illustrative embodiments. Accordingly, the particular system configurations and associated algorithm implementations can be varied in other embodiments.

A given processing device or other component of an information processing system as described herein is illustratively configured utilizing a corresponding processing device comprising a processor coupled to a memory. The processor executes software program code stored in the memory in order to control the performance of processing operations and other functionality. The processing device also comprises a network interface that supports communication over one or more networks.

The processor may comprise, for example, a microprocessor, an ASIC, an FPGA, a CPU, a TPU, a GPU, an ALU, a DSP, or other similar processing device component, as well as other types and arrangements of processing circuitry, in any combination. For example, at least a portion of the functionality of at least one neural network or an associated classification and/or remediation algorithm provided by one or more processing devices as disclosed herein can be implemented using such circuitry.

The memory stores software program code for execution by the processor in implementing portions of the functionality of the processing device. A given such memory that stores such program code for execution by a corresponding processor is an example of what is more generally referred to herein as a processor-readable storage medium having program code embodied therein, and may comprise, for example, electronic memory such as SRAM, DRAM or other types of random access memory, ROM, flash memory, magnetic memory, optical memory, or other types of storage devices in any combination.

As mentioned previously, articles of manufacture comprising such processor-readable storage media are considered embodiments of the invention. The term “article of manufacture” as used herein should be understood to exclude transitory, propagating signals. Other types of computer program products comprising processor-readable storage media can be implemented in other embodiments.

In addition, embodiments of the invention may be implemented in the form of integrated circuits comprising processing circuitry configured to implement processing operations associated with implementation of an AI-based classification algorithm utilizing one or more associated neural networks.

An information processing system as disclosed herein may be implemented using one or more processing platforms, or portions thereof.

For example, one illustrative embodiment of a processing platform that may be used to implement at least a portion of an information processing system comprises cloud infrastructure including virtual machines implemented using a hypervisor that runs on physical infrastructure. Such virtual machines may comprise respective processing devices that communicate with one another over one or more networks.

The cloud infrastructure in such an embodiment may further comprise one or more sets of applications running on respective ones of the virtual machines under the control of the hypervisor. It is also possible to use multiple hypervisors each providing a set of virtual machines using at least one underlying physical machine. Different sets of virtual machines provided by one or more hypervisors may be utilized in configuring multiple instances of various components of the information processing system.

Another illustrative embodiment of a processing platform that may be used to implement at least a portion of an information processing system as disclosed herein comprises a plurality of processing devices which communicate with one another over at least one network. Each processing device of the processing platform is assumed to comprise a processor coupled to a memory. A given such network can illustratively include, for example, a global computer network such as the Internet, a WAN, a LAN, a satellite network, a telephone or cable network, a cellular network such as a 4G or 5G network, a wireless network implemented using a wireless protocol such as Bluetooth, WiFi or WiMAX, or various portions or combinations of these and other types of communication networks.

Again, these particular processing platforms are presented by way of example only, and an information processing system may include additional or alternative processing platforms, as well as numerous distinct processing platforms in any combination, with each such platform comprising one or more computers, servers, storage devices or other processing devices. A given processing platform implementing an AI-based classification algorithm as disclosed herein can alternatively comprise a single processing device, such as a computer, a smartphone, a wearable device, an internal device, an intelligent medical monitoring device or handheld sensor device, that implements not only the classification algorithm and its associated one or more neural networks but also at least one data source and one or more controlled components. It is also possible in some embodiments that one or more such system elements can run on or be otherwise supported by cloud infrastructure or other types of virtualization infrastructure.

It should therefore be understood that in other embodiments different arrangements of additional or alternative elements may be used. At least a subset of these elements may be collectively implemented on a common processing platform, or each such element may be implemented on a separate processing platform.

Also, numerous other arrangements of computers, servers, storage devices or other components are possible in an information processing system. Such components can communicate with other elements of the information processing system over any type of network or other communication media.

As indicated previously, components of the system as disclosed herein can be implemented at least in part in the form of one or more software programs stored in memory and executed by a processor of a processing device. For example, certain functionality disclosed herein can be implemented at least in part in the form of software.

The particular configurations of information processing systems described herein are exemplary only, and a given such system in other embodiments may include other elements in addition to or in place of those specifically shown, including one or more elements of a type commonly found in a conventional implementation of such a system.

For example, in some embodiments, an information processing system may be configured to utilize the disclosed techniques to provide additional or alternative functionality in other contexts.

It should again be emphasized that the embodiments of the invention as described herein are intended to be illustrative only. Other embodiments of the invention can be implemented utilizing a wide variety of different types and arrangements of information processing systems, data sources, machine learning systems, machine learning models, neural networks, controlled system components and processing devices than those utilized in the particular illustrative embodiments described herein, and in numerous alternative processing contexts. In addition, the particular assumptions made herein in the context of describing certain embodiments need not apply in other embodiments. These and numerous other alternative embodiments will be readily apparent to those skilled in the art.

What is claimed is:

1. A method comprising:
obtaining an input data signal for a given individual;
generating a noisy version of the input data signal;
processing the noisy version of the input data signal in a denoising encoder-decoder neural network to generate a classification for the input data signal; and
executing at least one automated action based at least in part on the generated classification;
wherein the method is performed by at least one processing device comprising a processor coupled to a memory.

2. The method of claim 1 wherein the input data signal comprises at least one electrocardiogram (ECG) data signal obtained from one or more ECG sensors.

3. The method of claim 1 wherein the input data signal comprises at least one photoplethysmography (PPG) data signal obtained from one or more PPG sensors.

4. The method of claim 1 wherein the classification for the input data signal comprises a pain recognition classification providing a pain biomarker for the input data signal.

5. The method of claim 1 wherein the denoising encoder-decoder neural network comprises a deep artificial denoising auto-encoder-decoder (DADAED) neural network.

6. The method of claim 1 wherein the input data signal comprises at least a first data signal of a first type and a second data signal of a second type different than the first type, and further wherein noisy versions of the respective first and second data signals are generated and applied to respective first and second encoder-decoder pairs of the denoising encoder-decoder neural network for generation of respective first and second latent representations therefrom, the first and second latent representations being processed to generate the classification for the input data signal.

7. The method of claim 1 wherein the denoising encoder-decoder neural network comprises:
at least one encoder-decoder pair comprising an encoder and a decoder, the encoder having an input adapted to receive the noisy version of the input data signal and an output coupled to an input of the decoder;
an attention layer configured to process a latent representation generated by the encoder to generate a corresponding weighted representation; and
a classifier configured to receive the weighted representation from the attention layer and to generate therefrom the classification for the input data signal.

8. The method of claim 7 wherein the encoder-decoder pair is implemented using at least one artificial neural network (ANN).

9. The method of claim 7 wherein one or more parameters of the encoder and the decoder are selected to minimize error between the input data signal and a reconstructed version thereof generated by the decoder.

10. The method of claim 7 wherein the attention layer comprises:
a non-linear activation function configured to normalize received input; and
a softmax layer configured to process the normalized input to generate the weighted representation from the latent representation.

11. The method of claim 7 wherein the classifier comprises a multi-layer perceptron (MLP) classifier trained using a cross-entropy loss function.

12. The method of claim 1 wherein the input data signal comprises a first data signal of a first type and a second data signal of a second type different than the first type, and the denoising encoder-decoder neural network comprises:
a first encoder-decoder pair comprising a first encoder and a first decoder, the first encoder having an input adapted to receive a noisy version of the first data signal and an output coupled to an input of the first decoder, the first encoder generating a first latent representation from the noisy version of the first data signal;
a second encoder-decoder pair comprising a second encoder and a second decoder, the second encoder having an input adapted to receive a noisy version of the second data signal and an output coupled to an input of the second decoder, the second encoder generating a second latent representation from the noisy version of the second data signal;
an attention layer configured to process the first and second latent representations generated by the respective first and second encoders to generate at least one corresponding weighted representation; and
a classifier configured to receive the at least one weighted representation from the attention layer and to generate therefrom the classification for the input data signal.

13. The method of claim 12 wherein the first data signal of the first type comprises at least one ECG data signal and the second data signal of the second type comprises at least one PPG data signal.

14. The method of claim 1 wherein executing at least one automated action based at least in part on the generated classification comprises generating at least one output signal in a telemedicine application, wherein said at least one output signal in a telemedicine application comprises at least one of:
classification information for presentation on a user terminal or other display device;
classification information transmitted over a network to a medical professional; and
classification information transmitted over a network to a prescription-filling entity.

15. A system comprising:
at least one processing device comprising a processor coupled to a memory;
the at least one processing device being configured:
to obtain an input data signal for a given individual;
to generate a noisy version of the input data signal;
to process the noisy version of the input data signal in a denoising encoder-decoder neural network to generate a classification for the input data signal; and
to execute at least one automated action based at least in part on the generated classification.

16. The system of claim 15 wherein the input data signal comprises at least a first data signal of a first type and a second data signal of a second type different than the first type, and further wherein noisy versions of the respective first and second data signals are generated and applied to respective first and second encoder-decoder pairs of the denoising encoder-decoder neural network for generation of respective first and second latent representations therefrom, the first and second latent representations being processed to generate the classification for the input data signal.

17. The system of claim 15 wherein the denoising encoder-decoder neural network comprises:
at least one encoder-decoder pair comprising an encoder and a decoder, the encoder having an input adapted to receive the noisy version of the input data signal and an output coupled to an input of the decoder;
an attention layer configured to process a latent representation generated by the encoder to generate a corresponding weighted representation; and
a classifier configured to receive the weighted representation from the attention layer and to generate therefrom the classification for the input data signal.

18. A computer program product comprising a non-transitory processor-readable storage medium having stored therein program code of one or more software programs, wherein the program code, when executed by at least one processing device comprising a processor coupled to a memory, causes the at least one processing device:
to obtain an input data signal for a given individual;
to generate a noisy version of the input data signal;
to process the noisy version of the input data signal in a denoising encoder-decoder neural network to generate a classification for the input data signal; and
to execute at least one automated action based at least in part on the generated classification.

19. The computer program product of claim 18 wherein the input data signal comprises at least a first data signal of a first type and a second data signal of a second type different than the first type, and further wherein noisy versions of the respective first and second data signals are generated and applied to respective first and second encoder-decoder pairs of the denoising encoder-decoder neural network for generation of respective first and second latent representations therefrom, the first and second latent representations being processed to generate the classification for the input data signal.

20. The computer program product of claim 18 wherein the denoising encoder-decoder neural network comprises:
at least one encoder-decoder pair comprising an encoder and a decoder, the encoder having an input adapted to receive the noisy version of the input data signal and an output coupled to an input of the decoder,
an attention layer configured to process a latent representation generated by the encoder to generate a corresponding weighted representation; and
a classifier configured to receive the weighted representation from the attention layer and to generate therefrom the classification for the input data signal.
